(19) United States
(12) Patent Application Publication
(10) Pub. No.: US 2011/0263649 A1
(43) Pub. Date: Oct. 27, 2011

(54) CRYSTALLINE FORM OF LENALIDOMIDE AND A PROCESS FOR ITS PREPARATION

(75) Inventors: Priyanka Bhosale, Maharashtra (IN); Vinayak Gore, Maharashtra (IN); Ashok Pehere, Maharashtra (IN); Vinay Shukla, Maharashtra (IN)

(73) Assignee: GENERICS [UK] LIMITED, Potters Bar, Hertfordshire (GB)

(21) Appl. No.: 13/126,538

(22) PCT Filed: Nov. 2, 2009
(86) PCT No.: PCT/GB2009/051473

§ 371 Date: Jul. 12, 2011

(30) Foreign Application Priority Data
Nov. 3, 2008 (IN) 1870/KOL/2008

(51) Int. Cl. A61K 31/454; A61P 35/00; A61P 37/00; C07D 401/04; A61P 29/00

(52) U.S. Cl. 514/323; 546/200

(57) ABSTRACT

The present invention relates to a novel crystalline form of lenalidomide having formula (I) and chemically known as 3-(4-amino-1-oxo-1,3-dihydro-isoindol-2-yl)-piperidine-2,6-dione. The present invention further relates to a process for the preparation of said novel form and its use in pharmaceutical preparations for the treatment of autoimmune disease, inflammation, inflammatory disease and diseases such as cancer, in particular the management of multiple myeloma.

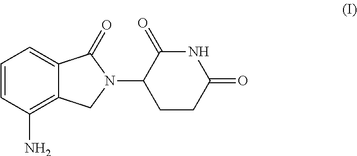

### FIELD OF THE INVENTION

The present invention relates to a novel crystalline form of lenalidomide having formula (I) and chemically known as 3-(4-amino-1-oxo-1,3-dihydro-isoindol-2-yl)-piperidine-2,6-dione. The present invention further relates to a process for the preparation of said novel form and its use in pharmaceutical preparations for the treatment of autoimmune disease, inflammation, inflammatory disease and diseases such as cancer, in particular the management of multiple myeloma.

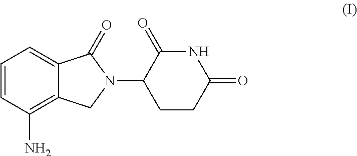

### BACKGROUND OF THE INVENTION

Lenalidomide is used in treating a wide range of disease including autoimmune disease, inflammation, inflammatory disease and cancer. Structurally, it is closely related to thalidomide.

U.S. Pat. No. 5,635,517 and U.S. Pat. No. 6,281,230 describe the preparation of lenalidomide and structural analogues. U.S. Pat. No. 5,635,517 relates to the use of lenalidomide to reduce undesirable levels of tumour necrosis factor α (TNFα). The lenalidomide is synthesized from 1,3-dioxo-2-(2,6-dioxopiperidin-3-yl)-4-nitro-isoindoline with catalytic hydrogenation at 50 psi hydrogen pressure and using 10% Pd/C suspended in 1,4-dioxane. The reduced product is filtered and, after removal of the solvent, the residue is crystallised from ethyl acetate to obtain an orange coloured product. The disclosed process results in a 69% yield. For further purification this solid is further recrystallised from a mixture of dioxane and ethyl acetate.

Both of the above-mentioned patents disclose methods of synthesis, isolation and crystallisation to increase the purity of lenalidomide. However, the patents do not disclose any polymorph or characterization data.

WO 2005/023192 and US 2005/0096351, both assigned to Celgene Corporation, disclose various crystalline forms of lenalidomide. The patent applications describe the preparation of the crystalline forms and characterise them using XRPD, Raman spectroscopy, and thermogravimetric methods such as DSC and TGA. In total eight crystalline forms (form A to form H) are reported, comprising hydrates and solvates and anhydrous forms. As claimed by the patentee, the hemi-hydrate crystalline form B is considered the desired polymorph of choice for formulation into a pharmaceutical product. Indeed form B has been used in the formulation of compositions for clinical studies. The various crystalline forms and some of their properties are summarised in table 1.

TABLE 1
Showing polymotphs of lenalidomide disclosed in WO 2005/023192

| Polymorph | Type | Preparation | Analysis |
|---|---|---|---|
| A | Anhydrous | Crystallisation in | XRPD, DSC, |
| | crystalline | non-aqueous solvent. | TGA, Raman |
| B | Hemi-hydrate | Slurrying | XRPD, DSC, |
| | crystalline | from various | TGA, Raman |
| | | solvents: hexane, | |
| | | toluene and water. | |
| C | Hemi-solvated | Slurrying from | XRPD, DSC, |
| | crystalline | acetone and then | TGA, Raman |
| | | slow cooling. | |
| D | Solvated | Crystallisation from | XRPD, DSC, |
| | crystalline | an acetonitrile:water | TGA, Raman |
| | | mixture. | |
| E | Dihydrated | Slurrying in water | XRPD, DSC, |
| | crystalline | or slow evaporation | TGA, Raman |
| | | in a solvent | |
| | | system comprising | |
| | | acetone:water (9:1). | |
| F | Anhydrous | Dehydration of | XRPD, DSC, |
| | crystalline | form E. | TGA, Raman |
| G | Anhydrous | Slurrying of forms | XRPD, DSC, |
| | crystalline | B and E in THF. | TGA, Raman |
| H | Partially hydrated | Exposure of | XRPD, DSC, |
| | crystalline | form E to 0% RH. | TGA, Raman |

The forms disclosed in WO 2005/023192 have poor aqueous solubility. The hemi-hydrate form B was prepared from an aqueous suspension of a polymorphic mixture. A suspension of lenalidomide was heated with 10 volumes of water at a temperature of 75° C. for 6-24 hours and then filtered at about the same temperature. The application clearly indicates that both starting material and the resultant form B are poorly soluble in water.

It is well known that poor solubility of an API can affect its bioavailability and also its dose size in a pharmaceutical formulation. In relation to dose size, a more soluble API means less API is needed to provide the same therapeutic effect compared with a less soluble API. This can of course be a factor in reducing any side effects that may be caused by the API. It is an aim of the formulation scientist to utilise forms of an API that provide the best solubility possible in order to reduce dosage size and minimise any side effect issues caused by large doses of an API and further to provide a composition with excellent bioavailability. Good solubility of an API in the development phase can help in reducing dose size.

WO 2005/023192 also explicitly discloses that form B is the most stable of all the disclosed crystalline forms in the presence of water or other aqueous systems. However, even form B is susceptible to conversion to another of the crystalline forms. A crystalline form that undergoes conversion in an aqueous environment is highly undesirable. Indeed it is a requirement of health authorities worldwide that the API in a pharmaceutical composition shows satisfactory polymorphic stability.

Thus it can be seen that although the various crystalline forms provide a repertoire of options for the formulation scientist, not one form is stable in terms of conversion to another crystalline form in an aqueous environment and adequate storage stability. For example, form A is stated as being the most thermodynamically stable, but it converts to form E in the presence of water or other aqueous solvent systems. Form B, stated as being the form of choice for use in pharmaceutical compositions, shows conversion to form E in aqueous solvent systems. Thus a pharmaceutical composition comprising form B would have to ensure that there was no ingression of water during storage or manufacture. This could be achieved by expensive packaging or by adding excipients such as a protective coating. This again adds to the cost of manufacture and further adds complexity to the manufacturing process.

The disclosure of WO 2005/023192 is rather confusing, as it states that form E is also the most stable in the presence of water or aqueous solvent systems. In any case the same arguments apply, whilst being one of the most stable crystalline forms in the presence of water or aqueous solvent systems, it also shows the greatest weight loss upon storage. Weight loss is indicative of degradation of an API.

In view of the above problems with the prior art, it can be seen that there is a need for a crystalline form of lenalidomide that has all the advantageous properties of polymorphic stability upon storage and during manufacture and in the presence of water and other aqueous solvent systems and desirable aqueous solubility.

In addition, it would be beneficial to develop an efficient method for interconversion of the newly reported polymorph to an existing polymorph having proven bioavailability, in particular form B, which has been designated as the form of choice by the applicants of WO 2005/023192 for pharmaceutical development.

### OBJECT OF THE INVENTION

A first object of the present invention is a novel polymorphic form of 3-(4-amino-1-oxo-1,3-dihydro-isoindol-2-yl)-piperidine-2,6-dione (lenalidomide), which is anhydrous and crystalline.

A second object of the present invention is a process for the preparation of the novel form of lenalidomide, wherein the process results in a good yield of the novel form according to the present invention.

A third object of the present invention is a new process for the preparation of existing form B, reported in WO 2005/023192, using the novel form of lenalidomide according to the present invention as an intermediate.

A fourth object of the present invention is a pharmaceutical composition containing the novel form of lenalidomide according to the present invention.

### SUMMARY OF THE INVENTION

Thus, according to a first aspect of the present invention there is provided an anhydrous crystalline form of lenalidomide having an X-ray powder diffraction pattern comprising a characteristic peak at about 46.6 or 46.7±0.2 degrees 2θ. A preferred embodiment provides an anhydrous crystalline form according to the invention having an X-ray powder diffraction pattern comprising further characteristic peaks at about 4.5, 22.7 and 32.4±0.2 degrees 2θ. A further preferred embodiment provides an anhydrous crystalline form according to the invention having an X-ray powder diffraction pattern substantially as shown in FIG. 1.

In another embodiment, an anhydrous crystalline form of lenalidomide is provided further characterised by showing little or no weight loss between 50-225° C. under thermogravimetric analysis (TGA) or alternatively further characterised by having a melting temperature at about 275° C. or 276° C.±5° C. as determined by differential scanning calorimetry (DSC).

In a second aspect of the present invention, a method for preparing an anhydrous crystalline form of lenalidomide according to the first aspect of the present invention is provided, comprising the steps of:

(a) dissolving or suspending lenalidomide in a solvent selected from the group comprising straight chained or branched C₁-C₅ alcohols, aliphatic ketones, and cyclic ethers or mixtures thereof;
(b) causing the desired crystalline form to precipitate; and
(c) isolating the resultant crystalline solid from step (b).

The lenalidomide used in step (a) may be crude. Preferably, in step (a), the lenalidomide is dissolved.

Preferably the solvent used in step (a) is a straight chained or branched C₁-C₅ alcohol, a straight chained or branched C₁-C₅ aliphatic ketone, a C₁-C₅ cyclic ether, or a mixture thereof. Preferably the alcohol is methanol, ethanol, propanol, isopropanol, n-butanol, or a mixture thereof. Preferably the ketone is acetone, butanone, methyl isopropyl ketone, or a mixture thereof. Preferably the cyclic ether is dioxane (such as 1,4-dioxane), THF, or a mixture thereof.

Preferably the C₁-C₅ alcohol is ethanol, or alternatively the aliphatic ketone is butanone or acetone, or in another alternative embodiment the cyclic ether is dioxane.

Optionally, in one embodiment, the solvent used in step (a) is not methanol. Optionally, in one embodiment, the solvent used in step (a) is not ethanol Optionally, in one embodiment, the solvent used in step (a) is not n-butanol. Optionally, in one embodiment, the solvent used in step (a) is not acetone. Optionally, in one embodiment, the solvent used in step (a) is not methyl ethyl ketone. Optionally, in one embodiment, the solvent used in step (a) is not THF. Optionally, in one embodiment, the solvent used in step (a) is not 1,4-dioxane.

In another alternative embodiment of the method according to the second aspect of the present invention, the lenalidomide is dissolved by heating the mixture from step (a) until a clear solution is obtained.

In a further preferred embodiment, the desired crystalline form is caused to precipitate by cooling the solution or suspension from step (a). Advantageously, the solution or suspension is cooled to between about 0-10° C.

In another preferred embodiment, the resultant crystalline solid from step (b) is isolated by filtration.

Preferably, the isolated solid is further washed with the solvent employed in step (a).

In yet another embodiment, the isolated solid is dried until a constant weight is achieved. Advantageously, the solid is dried at between about 40-60° C., most preferably between about 40-50° C. In a further embodiment, the solid is dried at between about 50-55° C. under conditions of reduced pressure for about 3-4 hours.

In a third aspect according to the present invention a method of converting the anhydrous crystalline form according to the first aspect of the present invention into prior art lenalidomide crystalline form B is provided, comprising the steps of:

(a) dissolving or suspending the anhydrous crystalline form of lenalidomide according to the first aspect of the present invention in a mixture of a polar organic solvent and water;
(b) causing the desired form B to precipitate from the solution or suspension in step (a); and
(c) isolating the solid crystalline form B.

Preferably, in step (a), the anhydrous crystalline form of lenalidomide is dissolved.

Preferably, the ratio of water to solvent is from about 5:95 to about 30:70. Most preferably, the ratio is about 20:80.

In a preferred embodiment, the polar organic solvent is a water miscible solvent, preferably a water miscible aliphatic alcohol. Preferred alcohols are methanol, ethanol, propanol, isopropanol, n-butanol, and mixtures thereof. In a particularly advantageous embodiment, the mixture is one of methanol:water or isopropanol:water.

In another alternative embodiment of the method according to the third aspect of the present invention, the lenalidomide is dissolved by heating the mixture from step (a) until a clear solution is obtained. Of course it will be understood that the lenalidomide can be fully dissolved or partially dissolved or even suspended and the method still fall within the scope of the invention. Accordingly, in a particularly advantageous embodiment, the mixture is heated to between about 45-55° C.

In a further preferred embodiment, crystalline form B is caused to precipitate by cooling the solution or suspension from step (a). Advantageously, the solution or suspension is cooled to between about 0-10° C.

In another preferred embodiment, the resultant crystalline solid from step (b) is isolated by filtration.

Preferably, the isolated solid is further washed with chilled methanol.

In yet another embodiment, the isolated solid is dried until a constant weight is achieved. Advantageously, the solid is dried at between about 50-60° C., most preferably between about 55-60° C. In a further embodiment, the solid is dried at between about 40-50° C. at about 100 mmHg pressure for approximately 3 hours.

In a preferred embodiment, the anhydrous crystalline form of lenalidomide according to the first aspect of the present invention or prepared by a process according to the second aspect of the present invention, is suitable for preparing other polymorphic forms of lenalidomide, in particular form B of lenalidomide.

In a preferred embodiment, the anhydrous crystalline form of lenalidomide according to the first aspect of the present invention or prepared by a process according to the second aspect of the present invention, or the crystalline form B prepared by a process according to the third aspect of the present invention, has a chemical and/or polymorphic purity of greater than about 90% respectively, more preferably greater than about 95%, more preferably greater than about 99%, and most preferably greater than about 99.5%. Chemical purity is preferably measured by HPLC. Polymorphic purity is preferably measured by XRPD, DSC and/or TGA.

In a preferred embodiment, the anhydrous crystalline form of lenalidomide according to the first aspect of the present invention or prepared by a process according to the second aspect of the present invention, or the crystalline form B prepared by a process according to the third aspect of the present invention, is suitable for use in medicine, preferably for treating inflammation, inflammatory disease, autoimmune disease or cancer.

A fourth aspect of the present invention provides a pharmaceutical composition comprising the anhydrous crystalline form of lenalidomide according to the first aspect of the present invention or prepared by a process according to the second aspect of the present invention, or comprising the crystalline form B prepared by a process according to the third aspect of the present invention, and one or more pharmaceutically acceptable excipients. In a particularly preferred embodiment, the composition is useful for the treatment of inflammation, inflammatory disease, autoimmune disease or cancer.

A fifth aspect of the present invention provides the use of the anhydrous crystalline form of lenalidomide according to the first aspect of the present invention or prepared by a process according to the second aspect of the present invention, or the use of the crystalline form B prepared by a process according to the third aspect of the present invention, in the manufacture of a medicament for the treatment of inflammation, inflammatory disease, autoimmune disease or cancer.

A sixth aspect of the present invention provides a method of treating inflammation, inflammatory disease, autoimmune disease or cancer, the method comprising administering to a patient in need thereof a therapeutically effective amount of the anhydrous crystalline form of lenalidomide according to the first aspect of the present invention or prepared by a process according to the second aspect of the present invention, or of the crystalline form B prepared by a process according to the third aspect of the present invention. Preferably, the patient is a mammal, preferably a human.

### BRIEF DESCRIPTION OF THE FIGURES

FIG. 1 shows an XRPD trace of the novel anhydrous crystalline form of lenalidomide according to the first aspect of the present invention.

FIG. 2 shows a differential scanning calorimetry (DSC) trace of the novel anhydrous crystalline form of lenalidomide according to the first aspect of the present invention.

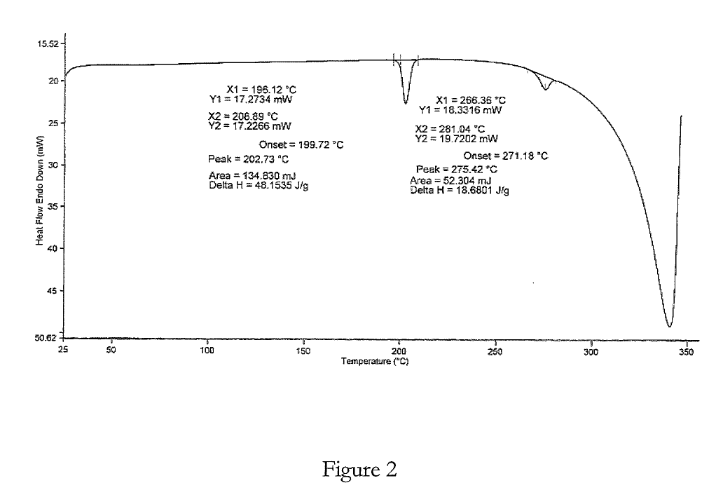

FIG. 3 shows a thermogravimetric analysis (TGA) trace of the novel anhydrous crystalline form of lenalidomide according to the first aspect of the present invention.

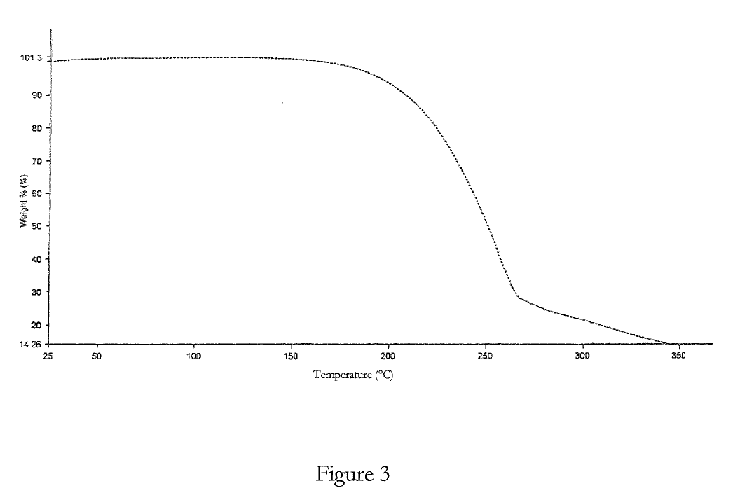

### DETAILED DESCRIPTION OF THE INVENTION

As used herein, the terms “polymorph”, “polymorphic form” and “crystalline form” are used interchangeably.

Lenalidomide has one chiral centre and therefore exists in two isomeric forms, the R-isomer and the S-isomer. The present invention encompasses racemic mixtures of the two isomers of lenalidomide as well as enantiomerically enriched and substantially enantiomerically pure isomers of lenalidomide. For the purposes of this invention, a “substantially enantiomerically pure” isomer of lenalidomide comprises less than 5% of the other isomer of lenalidomide.

The present invention provides a novel solid anhydrous crystalline form of lenalidomide and a process for its preparation. The process disclosed is simple and amenable to scale up and is capable of providing the polymorph in consistent crystalline and chemical purity of greater than 95% respectively, preferably greater than 96%, more preferably greater than 97%. Particularly preferred is a purity of greater than 98% and most preferred is a purity of greater than 99% irrespective of the scale of preparation.

Further, the novel crystalline form according to the first aspect of the present invention or prepared by a process according to the second aspect of the present invention provides a new polymorph which has an advantageous combination of features not previously described in the prior art. These features are:

(1) Polymorphic stability in the presence of water and aqueous solvent systems.
(2) Stability under normal storage conditions, i.e. storage at room temperature, taken as between about 20-30° C., preferably between about 25-28° C.
(3) High solubility in aqueous medium correlating to an increased excellent bioavailability and preferential dosing in a relevant pharmaceutical composition. Form B as disclosed in WO 2005/023192 and the polymorphic mixture from which it is obtained were prepared by the inventors and both were found to have poor aqueous solubility. Thus it is indeed advantageous to provide a polymorphic form of lenalidomide having a greater aqueous solubility as is provided by the first aspect of the present invention.

A process for the preparation of the polymorph according to the first aspect of the present invention comprises the following steps:

(a) dissolving or suspending crude lenalidomide in a solvent selected from the group comprising straight chained or branched C₁-C₅ alcohols, aliphatic ketones, and cyclic ethers or mixtures thereof;
(b) causing the desired crystalline form to precipitate; and
(c) isolating the resultant crystalline solid.

Of course it will be understood that the crude lenalidomide may be any crystalline form including those disclosed in the prior art. In preferred embodiments according to the invention, the lenalidomide is dissolved by heating the mixture from step (a) until a clear solution is obtained. This ensures that all of the crude lenalidomide is dissolved and generally leads to a purer end product. Of course it will be understood that the crude lenalidomide can be fully dissolved or partially dissolved or even suspended and the method still fall within the scope of the invention. Dissolution may also be effected by other techniques known in the art such as sonication or agitation or simply stirring.

The mixture may then be cooled in preferred embodiments to precipitate the desired crystalline form or in alternative embodiments an anti-solvent may be added. In preferred embodiments, the mixture is cooled to between about 0-10° C., most preferably to between about 0-5° C. The resultant precipitated solid can further be washed in the same solvent as utilised in step (a). A dry solid may be isolated by any means, preferably by vacuum filtration.

In particularly preferred embodiments, the isolated solid may be dried, preferably in conditions that do not induce conversion or degradation of the isolated solid. The inventors have found that drying under conditions of reduced pressure, preferably in a vacuum oven at between about 40-60° C., preferably about 50-55° C., at about 100 mmHg pressure for approximately 4 hours or until a constant weight is obtained, is particularly advantageous.

The inventors have found that preparation of the desired crystalline form of lenalidomide as described above results in lenalidomide having increased crystalline/polymorphic purity of greater than 99%. In further embodiments, crystallisation may be effected from solvents such as ethanol also resulting in lenalidomide having increased crystalline/polymorphic purity of greater than 99%.

Another aspect according to the invention provides that the novel polymorph can be transformed into other crystalline forms, in particular form B, which appears to be the crystalline form of choice to prepare pharmaceutical compositions with.

Accordingly, in a third aspect according to the present invention, a method of converting the anhydrous crystalline form according to the invention into prior art crystalline form B is provided, comprising the steps of:

(a) dissolving or suspending the anhydrous crystalline form of lenalidomide according to the first aspect of the present invention in a mixture of a polar organic solvent and water;
(b) causing the desired form B to precipitate from the solution or suspension in step (a); and
(c) isolating the solid crystalline form B.

Preferably, the polar organic solvent is selected from the group comprising water miscible aliphatic alcohols, typically lower aliphatic alcohols such as C₁-C₆ alcohols, most preferably methanol.

In particularly preferred embodiments, the mixture is preferably methanol:water or alternatively isopropanol:water.

In preferred embodiments, the volume of water in the solvent mixture can be between 10 to 30%, but is most preferably about 20%.

In preferred embodiments according to the third aspect of the present invention, the lenalidomide is dissolved by heating the mixture from step (a) until a clear solution is obtained. The inventors have found that heating to between about 45-55° C., most preferably between about 50-55° C., is most advantageous. This ensures that all of the crude lenalidomide is dissolved and generally leads to a purer end product. Of course it will be understood that the crude lenalidomide can be fully dissolved or partially dissolved or even suspended and the method still fall within the scope of the invention. Dissolution may also be effected by other techniques known in the art such as sonication or agitation or simply stirring.

The mixture may then be cooled to precipitate the desired crystalline form B or in alternative embodiments an anti-solvent may be added. In preferred embodiments, wherein the mixture from step (a) is heated to effect dissolution, the mixture may initially be cooled slowly to between about 25-30° C. The mixture may further be cooled, most preferably to between about 0-5° C. The resultant form B may, in preferred embodiments, be further washed with chilled methanol.

A dry solid may be isolated by any means, preferably by vacuum filtration. In particularly preferred embodiments, the isolated solid may be dried, preferably in conditions that do not induce conversion or degradation of the isolated solid. The inventors have found that drying in a vacuum oven at between about 45-55° C., preferably about 50-55° C., at about 100 mmHg pressure for approximately 4 hours or until a constant weight is obtained, is particularly advantageous.

In addition to the active ingredient(s), the pharmaceutical compositions of the present invention may contain one or more excipients. Excipients are added to the composition for a variety of purposes. Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulphate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol®), carboxymethyl cellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminium silicate, maltodextrin, methyl cellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethyl cellulose calcium, carboxymethyl cellulose sodium (e.g. Ac-Di-Sol®, Primellose®), colloidal silicon dioxide, croscarmellose sodium, crospovidone (e.g. Kollidon®, Polyplasdone®), guar gum, magnesium aluminium silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulphate, sodium stearyl fumarate, stearic acid, talc and zinc stearate.

Flavouring agents and flavour enhancers make the dosage form more palatable to the patient. Common flavouring agents and flavour enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colourant to improve their appearance and/or facilitate patient identification of the product and unit dosage level.

In liquid pharmaceutical compositions of the present invention, the crystalline form of lenalidomide according to the invention or alternatively form B of lenalidomide prepared according to the invention and any other solid excipients are dissolved or suspended in a liquid carrier such as water, vegetable oil, alcohol, polyethylene glycol, propylene glycol or glycerine.

Liquid pharmaceutical compositions may further contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel or organoleptic qualities of the product and/or coat the lining of the gastrointestinal tract. Such agents include acacia, alginic acid, bentonite, carbomer, carboxymethyl cellulose calcium or sodium, cetostearyl alcohol, methyl cellulose, ethyl cellulose, gelatin, guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxy anisole and ethylenediaminetetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts. Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or a soft shell. The shell may be made from gelatin and optionally contain a plasticizer such as glycerine and sorbitol, and an opacifying agent or colourant. The active ingredient and excipients may be formulated into compositions and dosage forms according to methods known in the art.

A composition for tabletting or capsule filling may be prepared by wet granulation. In wet granulation, some or all of the active ingredient and excipients in powder form are blended and then further mixed in the presence of a liquid, typically water, that causes the powders to clump into granules. The granulate is screened and/or milled, dried and then screened and/or milled to the desired particle size. The granulate may then be tabletted, or other excipients may be added prior to tabletting, such as a glidant and/or a lubricant.

A tabletting composition may be prepared conventionally by dry granulation. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a uniform tablet without granules. Excipients that are particularly well suited for direct compression tabletting include microcrystalline cellulose, spray dried lactose, dicalcium phosphate dihydrate and colloidal silica. The proper use of these and other excipients in direct compression tabletting is known to those in the art with experience and skill in particular formulation challenges of direct compression tabletting.

A capsule filling of the present invention may comprise any of the aforementioned blends and granulates that were described with reference to tabletting, however, they are not subjected to a final tabletting step.

In further embodiments, the composition of the invention may further comprise one or more additional active ingredients.

The details of the invention, its objects and advantages are explained hereunder in greater detail in relation to non-limiting exemplary illustrations.

### EXAMPLES

### Example 1

### Preparation of anhydrous lenalidomide according to the first aspect of the present invention from 1,3-dioxo-2-(2,6-dioxopiperidin-3-yl)-4-nitro-isoindole

1,3-Dioxo-2-(2,6-dioxopiperidin-3-yl)-4-nitro-isoindole (10.0 g, 0.035 mol) was suspended in ethanol (120 ml) to which was added iron powder (9.6 g, 0.175 mol). Hydrochloric acid (18.5 ml) diluted with an equal volume of water was added to this mixture. The reaction mixture was heated to between about 65-70° C. and maintained at this temperature for between about 1½-2 hours. The reaction mixture was cooled and filtered through a Celite® pad and the resultant clear filtrate was concentrated. The pH of the filtrate was adjusted to between 7 to 8 with ammonia solution and the concentrated filtrate was further filtered through a Celite® pad. The filtrate was again concentrated under reduced pressure. The product was finally dissolved in ethanol (50 ml) and heated at 45-50° C. until a clear solution was formed. The solution was cooled to about 5° C. and again filtered. The filtered solid was dried at 50-55° C. at reduced pressure for 3-4 hours until a constant weight of the product was obtained. The process resulted in 6.7 g (75% yield) of the novel anhydrous crystalline form of lenalidomide according to the present invention as an off-white to pale yellow powder.

The chemical purity of the novel form of lenalidomide formed was found to be 99.6% as measured by HPLC. The water content, DSC and TGA analyses confirmed that the novel form was anhydrous, i.e. not a solvate or hydrate. The XRPD of the novel form showed that the novel form was free from other polymorphic forms.

TABLE 2
XRPD trace of novel anhydrous form prepared according to example 1

| S. No. | 2-Theta Value | I/Iₘₐₓ ratio |
|---|---|---|
| 1 | 4.52 | 6.1 |
| 2 | 22.68 | 19.6 |
| 3 | 32.41 | 100.0 |
| 4 | 40.01 | 7.5 |
| 5 | 46.60 | 14.5 |

### Example 2

### Conversion of the Anhydrous Crystalline Form of Lenalidomide According to the First Aspect of the Invention to Prior Art Form B of Lenalidomide

Anhydrous crystalline lenalidomide (1.5 g) prepared according to example 1 was dissolved in a mixture of methanol:water (80:20) (22.5 ml) by heating the reaction mixture at 45-50° C. until a clear solution was formed. This solution was slowly cooled to about 25-30° C. When the solution started to appear turbid, it was chilled to between about 0-5° C. and maintained whilst stirring at this temperature for 2-3 hours. The crystallised product was filtered, washed with cold methanol (3 ml) and vacuum filtered dry. The product was finally dried at 45-50° C. at 100 mmHg pressure for approximately 3 hours. The resulting dried solid was submitted for XRPD, DSC and TGA analysis, which confirmed that form B as disclosed in WO 2005/023192 had been prepared.

The chemical purity of the form B of lenalidomide formed was found to be 99.65% as measured by HPLC. The XRPD of the form B formed showed that the form B was free from other polymorphic forms.

A comparison of TGA and DSC characterisation data is shown in table 3. Although the TGA results for prior art forms F and G are not given, the DSC results clearly differ from those of the crystalline form according to the first aspect of the invention, indicating the claimed form is indeed novel.

TABLE 3
Comparison of TGA and DSC characterisation data
of polymorphs A-H and the crystalline form according
to the first aspect of the present invention

| Polymorph | TGA (° C.) | DSC endotherms (° C.) | | |
|---|---|---|---|---|
| Polymorph A | No wt. loss in range | 270.28 | — | — |
| | 50-225° C. | | | |
| Polymorph B | 3.10% wt. loss in | 146.16 | 268.32 | — |
| | range 125-150° C. | | | |
| Polymorph C | 10.02% wt. loss in | 150.26 | 269.39 | — |
| | range 125-155° C. | | | |
| Polymorph D | 6.75% wt. loss in | 122.39 | 269.55 | — |
| | range 100-150° C. | | | |
| Polymorph E | 11.96% wt. loss in two | 98.99 | 168.79 | 268.84 |
| | steps: 50-100° C. | | (exotherm) | |
| | and 150-155° C. | | | |
| Polymorph F | Not available* | 268.57 | — | — |
| Polymorph G | Not available* | 248.20 | — | — |
| Polymorph H | 1.66% wt. loss in | 87.41 | 268.67 | — |
| | range 50-100° C. | | | |
| Novel | No wt. loss in range | 202.73 | 275.42 | — |
| polymorph | 50-225° C. | | | |

*data based on disclosure of WO 2005/023192

All references, including publications, patent applications and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

It will be understood that the present invention has been described above by way of example only. The examples are not intended to limit the scope of the invention. Various modifications and embodiments can be made without departing from the scope and spirit of the invention, which is defined by the following claims only.

1-27. (canceled)

28. An anhydrous crystalline form of lenalidomide having an X-ray powder diffraction pattern comprising a characteristic peak at about 46.6±0.2 degrees 2θ.

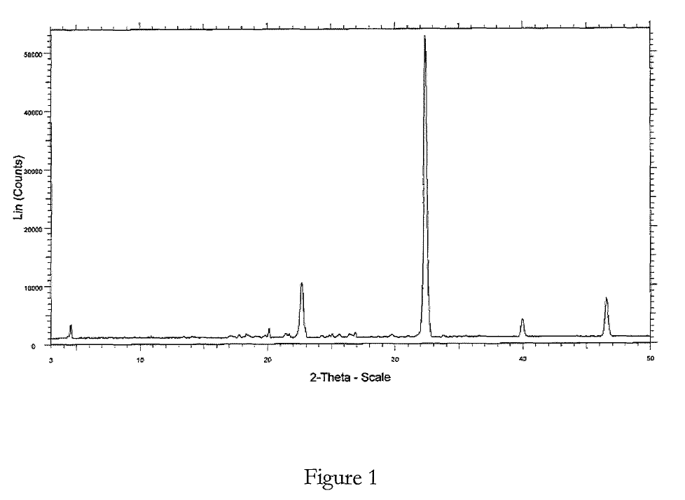

29. An anhydrous crystalline form according to claim 28:
(i) having an X-ray powder diffraction pattern comprising further characteristic peaks at about 4.5, 22.7 and 32.4±0.2 degrees 2θ; and/or
(ii) having an X-ray powder diffraction pattern substantially as shown in FIG. 1; and/or
(iii) further characterised by showing little or no weight loss between 50-225° C. under TGA; and/or
(iv) further characterised by having a melting temperature at about 275° C.±5° C. as determined by DSC.

30. A method for preparing an anhydrous crystalline form of lenalidomide according to claim 28, comprising the steps of:
(a) dissolving or suspending lenalidomide in a solvent selected from the group comprising straight chained or branched C₁-C₅ alcohols, aliphatic ketones, and cyclic ethers or mixtures thereof;
(b) causing the desired crystalline form to precipitate; and
(c) isolating the resultant crystalline solid from step (b).

31. A method according to claim 30, wherein:
(i) the C₁-C₅ alcohol is ethanol; and/or
(ii) the aliphatic ketone is butanone or acetone; and/or
(iii) the cyclic ether is dioxane.

32. A method according to claim 30, wherein:
(i) the lenalidomide is dissolved by heating the mixture from step (a) until a clear solution is obtained; and/or
(ii) the desired crystalline form is caused to precipitate by cooling the solution or suspension from step (a); and/or
(iii) the desired crystalline form is caused to precipitate by cooling the solution or suspension from step (a) to between about 0-10° C.; and/or
(iv) the resultant crystalline solid from step (b) is isolated by filtration.

33. A method according to claim 30, wherein the isolated solid is:
(i) washed with the solvent employed in step (a); and/or
(ii) dried until a constant weight is achieved; and/or
(iii) dried at between about 40-60° C.; and/or
(iv) dried at between about 50-55° C. under conditions of reduced pressure for about 3-4 hours.

34. A method of converting the anhydrous crystalline form according to claim 28 into crystalline lenalidomide form B, comprising the steps of:
(a) dissolving or suspending the anhydrous crystalline form according to claim 28 in a mixture of a polar organic solvent and water;
(b) causing the desired form B to precipitate from the solution or suspension in step (a); and
(c) isolating the solid crystalline form B.

35. A method according to claim 34, wherein:
(i) the ratio of water to polar organic solvent is from about 5:95 to about 30:70; and/or
(ii) the ratio of water to polar organic solvent is about 20:80; and/or
(iii) the polar organic solvent is a water miscible aliphatic alcohol; and/or
(iv) the solvent mixture is one of methanol:water or isopropanol:water; and/or
(v) the crystalline form is dissolved by heating the mixture from step (a) until a clear solution is obtained; and/or
(vi) the crystalline form is dissolved by heating the mixture from step (a) to between about 45-55° C.; and/or
(vii) the desired form B is caused to precipitate by cooling the solution or suspension from step (a); and/or
(viii) the desired form B is caused to precipitate by cooling the solution or suspension from step (a) to between about 0-10° C.; and/or
(ix) the resultant crystalline solid from step (b) is isolated by filtration.

36. A method according to claim 34, wherein the isolated solid is:
(i) washed with chilled methanol; and/or
(ii) dried until a constant weight is achieved; and/or
(iii) dried at between about 50-60° C.; and/or
(iv) dried at between about 40-50° C. at about 100 mmHg pressure for about 3 hours.

37. An anhydrous crystalline form of lenalidomide according to claim 28, having a chemical and/or polymorphic purity of greater than about 90%.

38. An anhydrous crystalline form of lenalidomide prepared by a process according to claim 30, having a chemical and/or polymorphic purity of greater than about 90%.

39. A crystalline form B of lenalidomide prepared by a process according to claim 34, having a chemical and/or polymorphic purity of greater than about 90%.

40. An anhydrous crystalline form of lenalidomide according to claim 28, for:
(i) use in medicine; and/or
(ii) treating inflammation, inflammatory disease, autoimmune disease or cancer.

41. An anhydrous crystalline form of lenalidomide prepared by a process according to claim 30, for:
(i) use in medicine; and/or
(ii) treating inflammation, inflammatory disease, autoimmune disease or cancer.

42. A crystalline form B of lenalidomide prepared by a process according to claim 34, for:
(i) use in medicine; and/or
(ii) treating inflammation, inflammatory disease, autoimmune disease or cancer.

43. A pharmaceutical composition comprising an anhydrous crystalline form of lenalidomide according to claim 28, and one or more pharmaceutically acceptable excipients.

44. A pharmaceutical composition according to claim 43, for the treatment of inflammation, inflammatory disease, autoimmune disease or cancer.

45. A pharmaceutical composition comprising an anhydrous crystalline form of lenalidomide prepared by a process according to claim 30, and one or more pharmaceutically acceptable excipients.

46. A pharmaceutical composition according to claim 45, for the treatment of inflammation, inflammatory disease, autoimmune disease or cancer.

47. A pharmaceutical composition comprising a crystalline form B of lenalidomide prepared by a process according to claim 34, and one or more pharmaceutically acceptable excipients.

48. A pharmaceutical composition according to claim 47, for the treatment of inflammation, inflammatory disease, autoimmune disease or cancer.

49. A method of treating inflammation, inflammatory disease, autoimmune disease or cancer, the method comprising administering to a patient in need thereof a therapeutically effective amount of an anhydrous crystalline form of lenalidomide according to claim 28.

50. A method according to claim 49, wherein:
(i) the patient is a mammal; and/or
(ii) the patient is a human.

51. A method of treating inflammation, inflammatory disease, autoimmune disease or cancer, the method comprising administering to a patient in need thereof a therapeutically effective amount of an anhydrous crystalline form of lenalidomide prepared by a process according to claim 30.

52. A method according to claim 51, wherein:
(i) the patient is a mammal; and/or
(ii) the patient is a human.

53. A method of treating inflammation, inflammatory disease, autoimmune disease or cancer, the method comprising administering to a patient in need thereof a therapeutically effective amount of a crystalline form B of lenalidomide prepared by a process according to claim 34.

54. A method according to claim 53, wherein:
(i) the patient is a mammal; and/or
(ii) the patient is a human.
